(19) United States
(12) Patent Application Publication
(10) Pub. No.: US 2022/0395374 A1
(43) Pub. Date: Dec. 15, 2022

(54) MOTION PRESERVATION BY AN ARTIFICIAL SPINAL DISC

(71) Applicant: TECHNION RESEARCH & DEVELOPMENT FOUNDATION LTD., Haifa (IL)

(72) Inventors: Or DAVID, Herzliya (IL); Moshe SHOHAM, Hoshaya (IL)

(21) Appl. No.: 17/603,939

(22) PCT Filed: Dec. 13, 2018
(86) PCT No.: PCT/IB2019/001299

§ 371 Date: Oct. 14, 2021

Related U.S. Application Data
(60) Provisional application No. 62/778,919, filed on Dec. 13, 2018.

(51) Int. Cl. A61F 2/30; A61F 2/44

(52) U.S. Cl. CPC A61F 2/30942; A61F 2/4425; A61F 2/447; A61F 2002/3037; A61F 2002/30387; A61F 2002/30471; A61F 2002/30537; A61F 2002/443

(57) ABSTRACT

A personalized intervertebral disc replacement for a subject includes a first element adapted to contact a first vertebra in the spine of the subject, a second element adapted to contact a second vertebra adjacent to the first vertebra in the spine of the subject, and a set of links coupling the first and second elements, the links arranged as a passive parallel mechanism, each of the links having a predetermined stiffness and length, and at least some of the links being oriented obliquely to a direction perpendicular to either of the first and second elements.

This application claims priority to U.S. Provisional Application No. 62/778,919 filed Dec. 13, 2018, the entire contents of each of which are hereby incorporated by reference.

### BACKGROUND

### 1. Technical Field

The field generally relates to surgical manipulation of the spine, especially for substitution of biological intervertebral discs by an artificial replacement.

### 2. Discussion of Related Art

Abnormalities of the cervical or lumbar spine resulting from aging or damage to the vertebrae may cause instability of the spinal column, resulting in neurological damage and pain. As intervertebral joints bear the axial weight of the body, the resulting forces predispose the intervening discs to damage over time. Surgical intervention to correct the resulting instability via fusion of the vertebrae above and below the affected disc often results in reduced movement of the neck or back, and due to abnormal strain on the contiguous vertebrae, may lead to further damage. Solutions to replace the affected intervertebral disk with an artificial replica have been only partially successful.

Historically, spinal fusion has been performed to surgically treat several spinal disorders, such as disc degeneration and spinal instabilities. With time, total disc replacement was developed to overcome functionality-loss-related post-surgical complications in the treated joint. In contrast to spinal fusion, total disc replacement allows motion of the treated vertebral level while preserving the original disc height. Several artificial discs have been developed through the years for this purpose. Although the long term effect of total disc replacement still needs to be studied, in follow-up periods of 2-5 years, it was proven to have a lower likelihood of adjacent level disease, complications and reoperation rates in comparison to the lumbar fusion technique.

While generally having better results than spinal fusion, total disc replacement has been reported as causing some post-operative complications, such as disc height loss, damaged facet joints, spontaneous fusion of the adjacent vertebrae, inflammations, mechanical wear, enlarged lordosis angle due to discectomy of ligaments during the surgery and other surgery-related problems.

Artificial discs are usually divided into two groups: semi-constrained devices, such as ball-and-socket bearings, like U.S. Pat. Nos. 5,314,477, 6,740,118 and U.S. Patent Application Publication No. 2007/0179615, and unconstrained devices that usually include compressible core or free-to-slide parts, such as U.S. Pat. Nos. 5,401,269, 5,556,431 and 5,071,437. The designation of unconstrained or semi-constrained relates to the design of the disc relative to kinematic motion. Discs with semi-constrained motion have elements that prevent the vertebral joint from moving freely in any direction. While semi-constrained devices have higher load sharing capabilities and reduced likelihood of hyper-motion, clinical studies have shown that the unconstrained devices have significantly lower sensitivity to placement errors, exert less pressure on the nearby discs and are less subjected to wear. Although natural motion can vary between patients, and can change over time with the presence of a pathological condition, remodeling of the intervertebral joints to match the new case creates a new kinematic profile that has to be followed.

While the physiological load capacities and range of motion of artificial discs are clearly defined and taken into account in the design process, their kinematic behavior is generally less addressed. Recent studies have shown that in many cases, disc arthroplasty results in significant changes of the segment's kinematics, leading to facet joint deformation, and subsequent back pain. Some recent developments, such as a spring based-design and the U.S. Pat. No. 5,071,437 artificial disc, try to cope with the kinematics problem. However, even though U.S. Pat. No. 5,071,437 provided good motion preservation, it has yielded poor clinical results, due to mechanical failure of its polyolefin core caused by material fatigue. Mechanical wear is common in most existing solutions, due to the high friction during relative motion between implant surfaces. The debris generated by wear on the disc is known to cause inflammations. Other motion preserving designs include the M6 artificial disc (U.S. Pat. No. 7,153,325) and European Patent No. 1,738,722, which only have documented clinical results in the cervical levels, where there are significantly lower loads than the lumbar level.

Recent prior art solutions for intervertebral disc replacements include U.S. Patent Application Publication No. 2018/0207002 to Glerum et al. for “Expandable fusion device and method of installation thereof;” U.S. Patent Application Publication No. 2018/0207000 to Zeegers for “Intervertebral disc prosthesis;” U.S. Patent Application Publication No. 2018/0098859 to Beaurain et al. for “Intervertebral disc prosthesis, surgical methods, and fitting tools.” A common factor in all these applications is that they provide a single size solution. They do not take into account the different anatomical and pathological status of the individual patient, nor how these factors might affect the successful integration and function of the prosthesis.

In U.S. Patent Application Publication No. 2017/0143502 to A. Yadin et al. for “Intervertebral Disc Replacement,” there is described, among other things, the use of a layer of numerous compressible column springs with various spring constants to mimic the behavior of a human disc.

U.S. Patent Application Publication No. 2018/0055654 to de Villiers et al., “Customized intervertebral prosthetic disc with shock absorption,” describes a plurality of choices for the core of the prosthesis in an attempt to customize the weight-bearing capacity and motion of the device. Chinese Patent No. 107736956 to Wang et al. for “Artificial intelligence cervical intervertebral disc capable of recording pressure and exercise” describes a device to provide feedback for rehabilitation training. PCT Application Publication No. WO2015010223 to Yang et al., “Apparatus and method for fabricating personalized intervertebral disc artificial nucleus prosthesis,” is a device for nucleus replacement. This device uses personalized information of the individual to be treated to determine the thickness and longitudinal and horizontal dimensions of a spiral-shaped artificial nuclear substitute.

Despite the many different models and attempts to build a physiologically compatible replica, prior art examples have drawbacks of various types. There therefore exists a need for a more physiological intervertebral disc replacement which preserves the natural dynamics of the spinal column of the individual patient and thereby overcomes at least some of the disadvantages of prior art systems and methods.

The disclosures of each of the publications mentioned in this section and in other sections of the specification, are hereby incorporated by reference, each in its entirety.

### SUMMARY

A personalized intervertebral disc replacement for a subject includes a first element adapted to contact a first vertebra in the spine of the subject, a second element adapted to contact a second vertebra adjacent to the first vertebra in the spine of the subject, and a set of links coupling the first and second elements, the links arranged as a passive parallel mechanism, each of the links having a predetermined stiffness and length, and at least some of the links being oriented obliquely to a direction perpendicular to either of the first and second elements.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

Further objectives and advantages will become apparent from a consideration of the description, drawings, and examples.

FIGS. 1A-1D show views of a spine under conditions of motion and an individual vertebra in superior and lateral views showing directions of motion.

FIG. 2 is a table showing predetermined loads for each motion.

FIG. 3 is a schematic view of a Stewart-Gough parallel mechanism with six degrees-of-freedom.

FIG. 4 is a configuration of the artificial disc having both upper and lower platform coordinates on a circle.

FIG. 5A is a schematic view of an adult adjacent pair of lumbar vertebrae with an exemplary artificial disc according to the present disclosure positioned between them, while FIG. 5B is a table showing the Young's modulus of some biocompatible materials which may be used for the links of the artificial disc of FIG. 5A.

FIG. 6A-6B are flowcharts showing the method of artificial disc design and the calculation of the Finite Screw Axis of the joints using a motion simulator.

FIG. 7 shows an isometric representation of a completely assembled artificial disc of the type shown in FIG. 5A.

FIG. 8A-8D illustrate how links can be attached to the platforms in such a way that the ball joints are held firmly within their sockets, while allowing free angular motion.

FIGS. 9A and 9B and FIGS. 10A, 10B, and 10C illustrate two alternative methods by which the platforms can be constructed in smaller parts which can be assembled in situ to generate a rigid plate.

FIG. 11 illustrates a parallel robot stiffness model.

FIG. 12 illustrates notations for a rigid body motion screw.

FIG. 13 illustrates a cost function calculation process for iteration j.

FIG. 14 illustrates a multi start global optimization scheme.

FIG. 15 illustrates a Stewart-Gough platform used for simulation.

FIG. 16 illustrates natural and artificial instantaneous twists for a spinal disc-like parallel robot with optimized actuator stiffnesses.

FIG. 17. is a schematic illustration of an equilateral triangular platform configuration and parameter definition.

FIG. 18 illustrates an intervertebral disc replacement that includes a semi-rigid nuclear body.

### DETAILED DESCRIPTION

The present disclosure describes a new exemplary artificial vertebral spinal disc for replacement of a defective intervertebral spinal disc that maintains the natural motion of the adjacent vertebrae. The structure of this artificial intervertebral disc joint is based on a passive parallel robot mechanism, with the robotic actuators replaced by passive links, where the lengths and the stiffnesses for each of the links between the two platforms of the mechanism may be different. The mechanism is constructed to resemble as closely as possible the natural kinematics of a disc joint, such that the resulting motion of the optimized artificial disc closely resembles the natural 3-D motion of the joint between the two adjacent vertebrae connected by their natural intervertebral disc. The lengths and stiffnesses of the artificial disc links determine the vertical height of the disc under load, and the allowable range of motion of the adjacent vertebrae in any given direction. The stiffnesses are determined for each patient based on a preoperative analysis of the subject's range of motion in each of the three primary directions, as can be derived from the patient's X rays in flexion/extension/lateral bending and rotation, in order to optimize the disc vertical dimensions and to preserve the natural motion of the affected spinal segment in forward/reverse and lateral bending, and in rotational motions. In case the subject's vertebral range of motion precludes defining these natural motions due to his/her pathological condition, e.g., scoliosis or other condition that restricts movement of the spinal column, these range of motion data are obtained from biomechanical atlases or can be designed according to the surgeon's preferences.

These three main motions of a normal human spinal column can be defined and quantified using principles of mechanical engineering such as the instantaneous axis of rotation (IRA) or instantaneous screw axis (ISA). In some cases, the finite screw axis (FSA) is used which, in the present case, is closely related to IRA.

To perform the required calculations for determining the IRA/ISA for any joint, radiographic images are taken of a patient's spine in the area of the degenerated disc needing replacement, for example the L4-L5 lumbar disc. One or more sets of images are acquired in resting position, either three dimensional images by CT or MRI, or plain x-ray images. If two dimensional x-ray images are used, both anterior-posterior and lateral views may be taken. To measure the displacement of the intervertebral disc during motion, further fluoroscope x-ray images should be taken in positions of right and left lateral bending, flexion, extension, and torsion. Measurements are made of the relevant disc dimensions in each of these positions. The FSA of each joint is determined for each position imaged during motion. The behavior of an artificial disc mimicking that of the natural disc, can then be simulated under a set of characteristic loads, and optimal stiffnesses for each joint can be calculated, based on inverse and forward kinematics. By comparing and optimizing the distance and angle between the natural and artificial FSA, the best combination of FSAs for the three primary back motions can be determined. Optimization of the artificial disc dimensions and link stiffnesses enables convergence of the FSA of the artificial disc joint, to that of a natural disc. As a result, the artificial disc preserves the natural motion of the adjacent vertebrae to a great extent, and thereby loads, e.g., loads on the facet joints, are minimized.

The present disclosure has several benefits over the existing designs. It incorporates a computational method that allows selection of various parameters, enabling a design of the best individual implant for a patient. The artificial disc described herein carries the spinal loads while closely preserving the natural motion of the intervertebral disc joint. The mechanism is constructed to resemble as closely as possible the natural kinematics of a given disc joint. As the parameters of each replacement disc are based on measurements taken from the patient close to the time of replacement, it is unique and personalized to the individual. As such, this system is the first example of personalized medicine with respect to intervertebral disc replacement.

Due to the high complexity of in vivo spinal load measurements, the artificial disc in the present disclosure mimics the characteristic behavior of a lumbar spinal disc, as measured in vitro. The natural behavior of the disc is evaluated using data from an in vitro study in which the neural arches and spinal ligaments were removed from the vertebral column. This is a valid system for the measurements needed, because, although large coupling magnitudes have been measured in vivo between neighboring vertebrae, it appears that these are caused by the spinal column as a whole, and not by the disc itself.

The artificial disc device is a derivative of a parallel robotic platform having six degrees of freedom, with the robotic actuators being replaced with passive links, each having individually calculated lengths and stiffnesses, selected to mimic the natural motion allowed by the disc joint. Such a mechanism provides the mechanically most economical means to obtain a given motion in all 3D axes with a minimal number of links. Thus, the device operates as a passive parallel mechanism, motion of which is determined by the set of link configurations and their stiffnesses. This term “passive parallel mechanism” is used herein to describe such a mechanical configuration, and is also thuswise claimed. A particularly convenient platform for achieving these aims is the Stewart-Gough platform, though the device is not intended to be limited to this configuration. The base of the device may be considered to be stationary (though it is, of course, the mutual motion between base and upper platform that is important in the design) and may conveniently be triangular or circular, but is not limited to these geometric shapes. The top of the platform generally, but not necessarily, has a flat geometric design and moves passively, relative to the base, with the displacement of each leg. The platform and links are composed of biocompatible material.

The interior of the personalized intervertebral disc replacement may include a semi-rigid nuclear body comprising inert biocompatible material having a specific pre-calculated resistance for the subject.

The stiffness of each link is calculated by using a combination of inverse and forward kinematics analysis. The method involves selecting the desired stiffness with respect to the natural motion, characterized by FSA.

Feasibility check of relevant bio-compatible materials for the mechanism's links showed that they do not buckle under physiological loads due to motion of spine vertebrae. The structure of the artificial disc can be further modified to minimize facet joints displacement or any other displacement parameter, as a cost function.

The artificial disc of the current disclosure addresses several common problems in the prior art artificial discs. The device and method of the current disclosure allows different discs to be designed for different clinical scenarios. For example, a different model would be suitable in a patient with a degenerated disc, which requires remodeling of all of the adjacent vertebral joints, as opposed to a patient with scoliosis. In scoliosis, the term “natural motion” or “normal curvature” does not relate to the parameters discussed here, but to a set of parameters that would restore the desired kinematic behavior in the best possible manner. Thus, in patients with limited motion of the spinal column, artificial discs with calculated natural motion could be used to provide improved mobility. In these cases, the calculations for natural motion could be taken from databases comprising such information gathered from normal individuals with similar physiological parameters, such as gender, age, height, weight, and body-mass index. Big data or artificial intelligence can be applied to such calculations to produce the optimal configuration for a given patient.

Finally, the use of a Stewart-Gough platform-based design also allows the possibility to implant the device in a minimally-invasive procedure, due to the small size of the components. In some implementations each component of the structure can be inserted separately and assembled in situ during the operation. In such cases, the upper and lower elements and links are configured to allow them to be inserted individually into a patient intraoperatively and assembled in situ, thereby minimizing surgical trauma to the patient and allowing more rapid recovery.

The artificial disc is designed as a load-bearing device, the top and bottom platforms and center of which are designed to support the compressive load of the entire upper body. The center of the artificial disc may be comprised of inert biocompatible material having a specific pre-calculated resistance. In some implementations, the artificial disc is contained within a flexible outer covering of a biocompatible material.

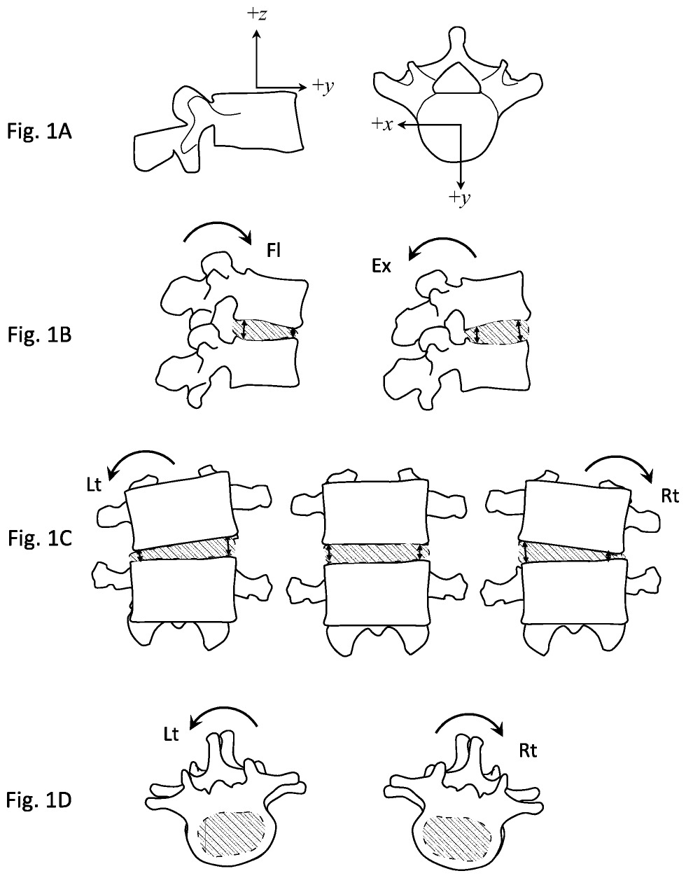

Reference is now made to FIGS. 1A-1D, which illustrate schematically human vertebrae and their relative directions of movement in three dimensions. FIG. 1A shows side and top views of a single vertebra, in this example, from the lumbar region, with the three relative axes of motion labeled. FIGS. 1B-1D illustrate the various positions used to measure disc displacement during the respective motions and thereby calculate the link stiffnesses. Flexion/extension (F/E) (FIG. 1B) are rotations about the x-axis, lateral bending (LB) (FIG. 1C) is rotation about the y-axis, and axial torsion (AR) (FIG. 1D) is rotation about the z-axis. In FIG. 1B, the curved arrows represent flexion (Fl) or extension (Ex). In FIGS. 1C and 1D, relative movement to the right (Rt) or left (Lt) is indicated by a curved arrow in that direction. The small double-headed arrows in FIGS. 1B-1C represent the disc height with the subject at rest or in a pose resulting from a specific motion, as indicated in the drawing. These views are meant to provide an example of the medical images, usually two dimensional x-ray images of the patient's spine, obtained by the physician preoperatively. Such images are used to calculate the relevant displacements of the intervertebral disc from a resting position for each motion.

Some of these motions are coupled, such that movement occurs along two axes simultaneously. The accompanying translations/rotations during human movements have been measured in vivo, as have the lordosis angle and initial position of each vertebra under conditions of normal spinal anatomy and physiology. Thus, information on structure and function of normal vertebrae under static and dynamic conditions is available and can be used to calculate force parameters as will be described below.

In addition to data available in the medical literature, the individual patient's medical images may be used to calculate spinal parameters such as lumbar lordosis, thoracic kyphosis, pelvic tilt, sagittal vertical axis, and pelvic incidence. The spinal parameters of the patient's spine preoperatively may be optimal, or due to spinal pathology, may be abnormal. Generally, the desired spinal parameters of the corrected anatomy are determined by the physician when making a pre-operative surgical plan. These corrected spinal parameter values may be taken into account when designing the artificial disc, such that the final height and stiffness of the artificial disc result in the desired corrected parameters. Whereas the examples in this drawing and further drawings described below pertain to the lumbar spine, similar measurements and calculations could be applied to other segments of the spinal column for the purpose of generating an artificial disc suitable for those positions.

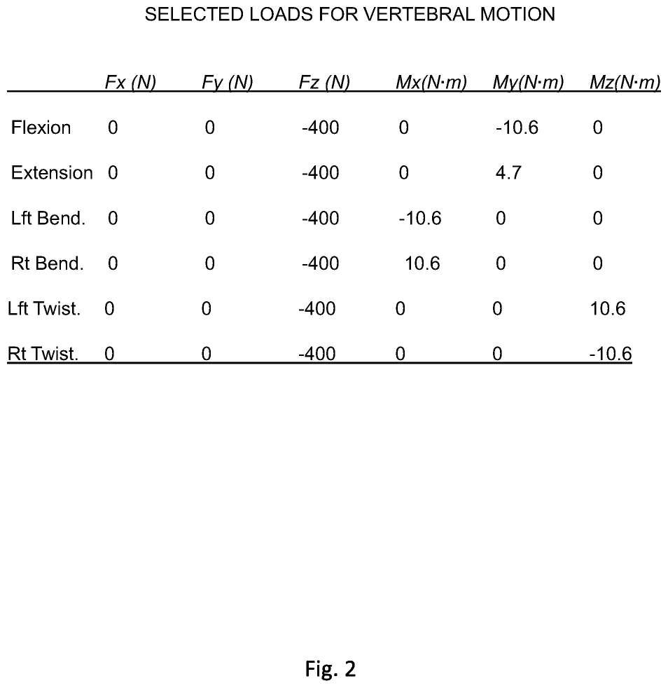

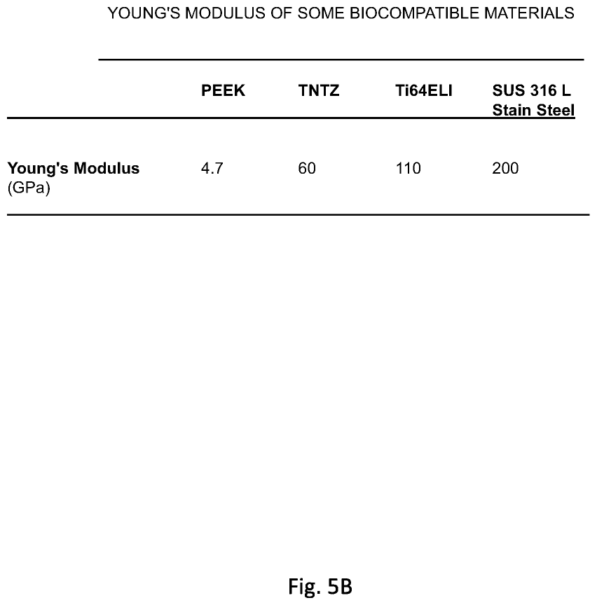

Reference is now made to FIG. 2, which is a table compiled from the literature, showing a set of loads for each motion, using the calculated magnitudes of coupling in two axes, based on known motions of vertebrae under normal conditions. The calculations in this example were compiled from previous publications in the field that determined loads on spinal discs, including Schultz et. al, “Mechanical Properties of Human Lumbar Spine Motion Segments—Part I: Responses in Flexion, Extension, Lateral Bending, and Torsion,” J. Biomech. Eng., vol. 101, no. 1, pp. 46-52, February 1979. These loads comprise a combination of pre-compression (Fx,y,z) and pure moments (Mx,y,z) as shown in FIG. 2, and are used to calculate displacements for the artificial disc to be described below in FIGS. 6A and 6B. From the data presented in FIG. 2 or from other sources, it is possible to estimate load on the spinal joints. Kinematic changes in the functional spinal unit, i.e., the entire spinal column, can be characterized by the FSA, which is an efficient parameter for comparison of natural versus pathological movements. Such use of the FSA can also provide an indication of facet joint internal forces.

It is to be understood that, whereas the values in FIG. 2 are representative for human lumbar spine motion, the surgeon may decide to use other load values extrapolated from an individual patient's characteristics, such as height, weight, body-mass index, and vertebral pair to be operated. Such load values may also be refined and updated over time using the long-term success of discs with a given set of stiffnesses in many patients, and inputting this information into a database for future reference.

Simulating the artificial disc behavior under a set of characteristic loads, and comparing and optimizing the distance and angle between the natural FSA, and that of the derived artificial disc configuration, can provide the best combination of FSAs for the artificial disc for performing the three primary back motions. To obtain the optimal FSA set for a given vertebra, it is advantageous to apply relative importance or weights to the three primary back motions, since they may contribute differently to the subject's pain condition.

In such an artificial vertebral joint, the relative motion of adjacent facet joints is significant, as this motion is considered to contribute much of the pain in the pathological spine. Thus, motion at the adjacent facet joints can be taken into account to enable verification of the facet displacements after the link stiffness calculations are performed. Adjustments can be made to the link stiffnesses to take into account facet displacements based on the contribution of facet pain to the individual patient's condition, as determined preoperatively.

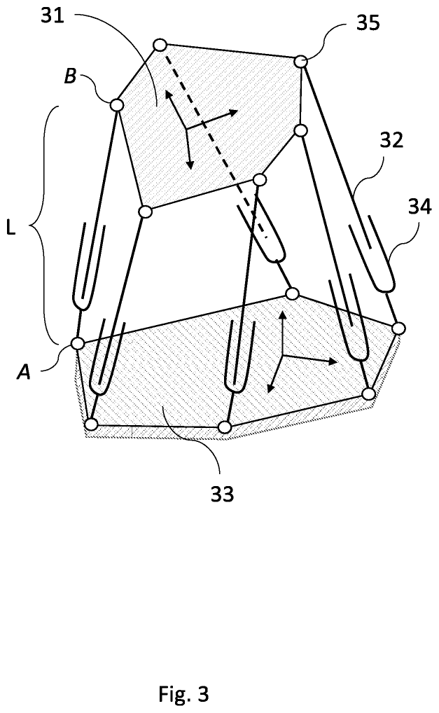

Reference is now made to FIG. 3, which shows a schematic representation of an artificial disc of the current disclosure in the form of a six-degrees-of-freedom Stewart-Gough platform having extendable legs 32 with extension links 34 and spherical joints 35. The distance between the movable platform 31 and the base 33 is measured at each leg or link 32, to provide a rest state length L between points A and B. These measurements are performed for each leg under static conditions, which gives a distance defined as the resting length, and then under three common motions: flexion/extension (FE), axial rotation (AR) and lateral bending (LB). From the measurements of the lengths of each leg under each condition of motion, the displacement dᵢ can be calculated, where d is the difference in length for each leg i, between the static position and the position for any given motion. For example, if at rest a leg had a length of 8 mm, and in a position of lateral bending it had a length of 10 mm, the displacement would be 2 mm. Subsequently, using a Jacobian matrix and inverse kinematics, the relevant link force can be calculated for each type of motion as described in below in FIGS. 6A, 6B.

The actuated linear joints 34-32 may be replaced with one solid rod with a given elasticity thus having a passive mechanism. In order to obtain the natural motion of the adjacent vertebra each link can be designed with different length and different elasticity.

The artificial disc is designed as a load-bearing device, the top and bottom platforms and center of which are designed to support the compressive load of the entire upper body. The center of the artificial disc may be comprised of inert biocompatible material having a specific pre-calculated resistance. Calculations of expected artificial disc behavior compared with that of the natural disc are carried out assuming a 400 N compression load imitating the force of the human torso, as shown in FIG. 2. The relative importance of each of the three spinal motions is weighted individually. For example, assuming the main cause of spinal pain in a given patient is axial rotation, then its relative weight in calculating the optimized displacement of the adjacent vertebra would be set at twice that of the other motions. Platform shapes of the top and bottom surfaces of the artificial disc may be either triangular or circular or any other regular, non-planar shape.

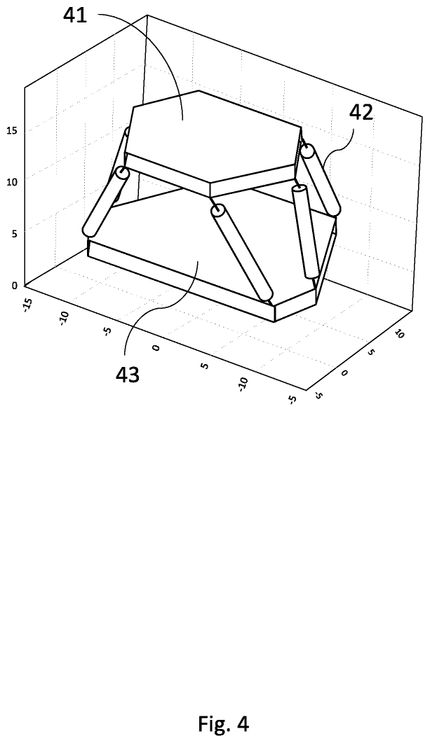

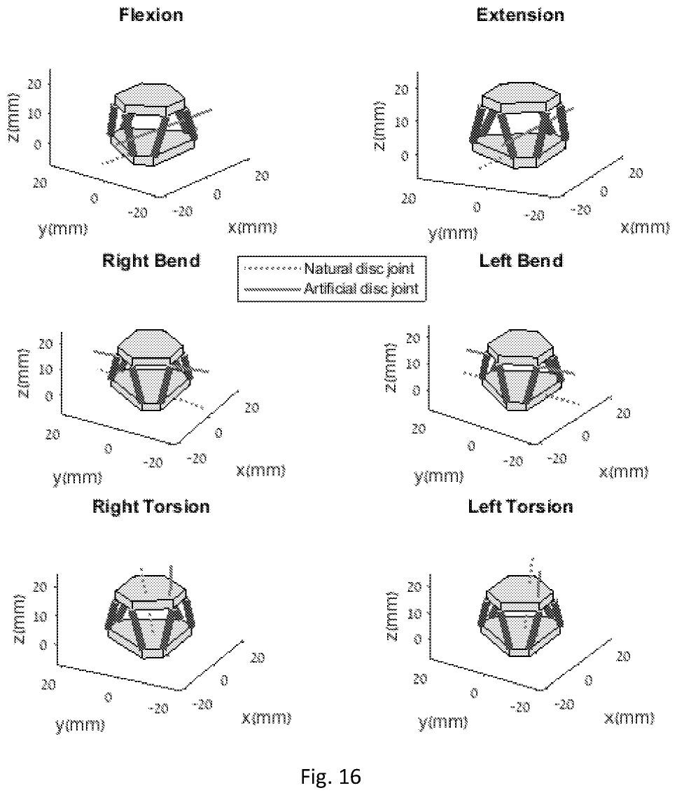

Reference is now made to FIG. 4, showing schematically the dimensions of a typically optimally configured artificial disc using the optimization procedure of the present disclosure. This disc is optimized for one specific case of weighted motion using loads as given in FIG. 2. The numbers along the edge of each axis represent the dimensions in millimeters. Simulation of the L4/L5 disc performance can be performed assuming the main cause of pain is axial rotation (AR). For the sake of simplicity in such calculations, top and bottom surfaces of the artificial disc, 41 and 43, are defined as rigid, planar equilateral triangles, and the positions of the links 42 can be defined by the distance of each connection point from the center of the relevant edge of upper and lower surfaces. FIG. 17. is a schematic illustration of an equilateral triangular platform configuration and parameter definition. Parameter xᵢ defines the distance of each point from the center of each side. For stiffness calculations, the relative weighting of AR is set at double the weighting chosen for the other motions (FE and LB). A cost function which presents the misalignment of the natural and the artificial FSA is defined. The problem therefore becomes a problem of minimizing the cost function, which yields a 2D relative position and orientation of the natural vs. the artificial FSA optimization problem.

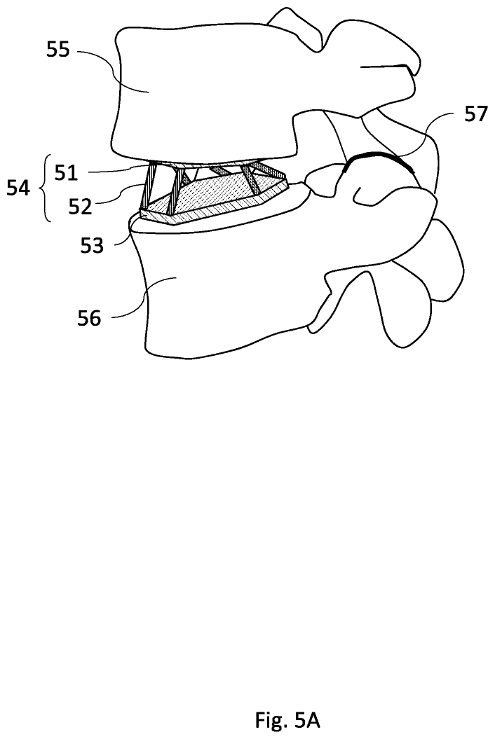

Reference is made to FIG. 5A, presenting an artificial disc 54 for positioning between two adjacent vertebrae 55, 56. The surfaces of the disc contacting the adjacent vertebrae may have ridges or other elements that enable the disc to be firmly situated between the vertebral body end faces. The platforms 51, 53 may comprise any convenient biocompatible material having the required strength. For simplicity, each platform is considered planar, and all connection points lie on a triangle or circle as shown in FIGS. 4 and 17. However, it is to be understood that the platforms may also comprise shapes such as wedges or other non-parallel designs. Furthermore, the surfaces need not be planar; they may be contoured to the shape of the adjacent vertebral bodies or may comprise any other element that facilitates situating the artificial disc in the intervertebral space. The linear actuators at each link 52 of a common Stewart-Gough parallel robot are omitted, resulting in a passive mechanism, the overall motion of which is determined by the set of link configurations and stiffnesses. For a typical adult-sized lumbar disc replacement, the mechanism generally includes six links 52, each usually having a circular cross-section, with a diameter in the range of 3-5 mm. Typical dimensions for the upper 51 and lower 53 platforms are for lateral width 30-40 mm, anterior-posterior width 25-30 mm, and thickness 3 mm. The facet joint 57 between the two vertebrae 55, 56, is subject to movement during positions of flexion, extension, lateral bending and axial rotation.

Taking into account that the upper and lower platforms have a known thickness, the total height of the disc is determined by the length of each link, and its angular orientation. The average distance between two adjacent vertebrae in the lumbar region of the adult spine is 12 mm, an average which is dependent on patient age, gender, specific vertebral pair and possibly other health-related parameters. Given that an artificial disc replacement is needed because the patient's natural disc is ruptured or otherwise diseased, the current height of the patient's intervertebral space may not be the same as that desired for the replacement disc. Thus, when determining the length for each link of a replacement disc for a particular patient, the following parameters may be taken into consideration: measured height of the intervertebral space across the width and depth of the adjacent vertebrae on the current patient medical images; the measured heights in the same position from previous medical images of this patient, if available; the surgeon's past experience with disc replacements; the patient's age and gender, and a database of normal disc heights from healthy subjects of the relevant age and gender.

The links should be composed of biocompatible material having some compressibility. One measure of the ability of a material to withstand changes in length when under lengthwise tension or compression is the modulus of elasticity, or Young's modulus, given by the longitudinal stress divided by the strain. Examples of the Young's modulus of some biocompatible materials are given in FIG. 5B. The link stiffnesses are calculated according to the flowcharts presented and described in FIGS. 6A and 6B below.

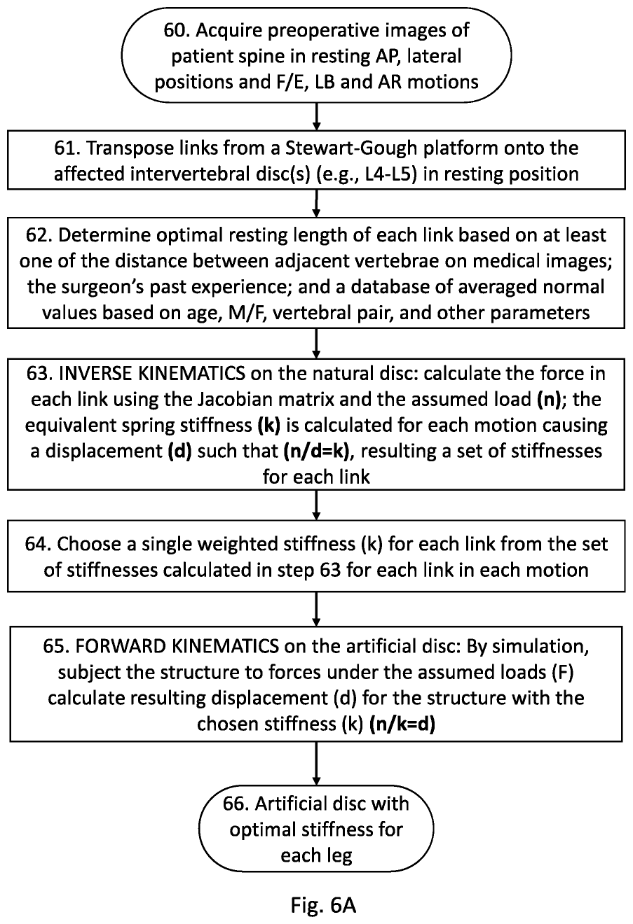

The presently described discs, in order to resemble as much as possible, the natural disc joint motion in all three dimensions, use the minimum numbers of flexible links (six) and in an oblique orientation relative to the upper and lower platforms, and hence also to the adjacent vertebral bodies. The current design is adaptable for a minimally invasive approach as it contains few parts, allowing the disc components to be inserted piece by piece through a small incision and assembled in situ, as will be described in connection with FIGS. 7 to 10 hereinbelow. Herein is disclosed a method for calculating, with a high degree of accuracy, a specific and potentially different stiffness for each link, a procedure which is not described in the prior art.

Reference is now made to FIG. 6A, which shows a flowchart delineating the steps of the inverse and forward kinematics calculations performed in order to determine the configuration of the links of the artificial disc.

In step 60, preoperative images of the patient spine are acquired at the level where the disc replacement is required, preferably in the upright resting anterior-posterior and lateral positions, and in poses of flexion, extension, lateral bending to left and right, and axial rotation/torsion in both directions. The disc position can be the commonly problematic lumbar L4-L5 intervertebral space or any other spinal level.

Next, the schematic view of the parallel mechanism, e.g. a Stewart-Gough platform, is virtually added to the images, and in step 61, the six links from the Stewart-Gough robotic assembly as described in FIGS. 3-5, are transposed onto an image of the affected intervertebral disc joint(s) in the upright resting position of the spine.

In step 62, the resting length of each link is calculated from measurements of the intervertebral disc height at various locations on the medical images with the patient in the upright resting pose. The optimal resting length of each link is determined based on at least one of the distance between adjacent vertebrae on medical images; the surgeon's past experience; and a database of averaged normal values based on age, gender, vertebral pair, and other parameters. The relative displacement dᵢ of each link for each movement can be easily calculated by comparing the resting lengths with the length in each position of motion. Thus, step 62 will result in a set of six displacement values for each of the six links, based on the six motions, which are flexion, extension, right lateral bending, left lateral bending, axial torsion to the right and axial torsion to the left.

In step 63, using inverse kinematics, the force (n) on each link is calculated as will be shown in the block flowchart of FIG. 6B, using the assumed loads on the entire disc from the table in FIG. 2, and the measurements of link displacements in step 62, and the angle of orientation of the link. From these values, the equivalent spring stiffness (k) is calculated from the known relation n/d=k, for each link in the case of each motion. The result of step 63 is then a matrix of six stiffness values, for each of the six links.

In step 64, the set of stiffness values derived in step 63 is used to select a single, e.g. an “averaged” stiffness (k) for each link from the set of stiffnesses calculated in each motion pose for a given link in step 63. The single “averaged” k value selected is based on a weighted average of the stiffnesses calculated for the three types of motion, F/E, LB, AR, such that any specific motion may be given more relative weighting. The determination of whether to weight each motion equally, or to give a given type of motion more importance, may be made by the surgeon based on considerations specific to the patient under treatment. For example, in a typical implementation, axial rotation or torsion may be weighted double that of the other two types of motion, if axial rotation is the main cause of the pain to be treated by the planned procedure.

In step 65, forward kinematics is used to calculate the resulting displacement (d) for each of the links with the chosen stiffness (k) for the artificial disc being designed for a given patient. Computer simulation is used to apply force to the disc under the assumed loads, i.e., using the load values from FIG. 2 and step 63, and using the weighted k value determined in step 64. The resultant displacement dᵢ values are compared with those of the native disc to ensure that the disc design provides for normal spinal motions, mimicking to the greatest extent those allowed by the natural disc it is meant to replace. This step is necessary in order to ascertain that the designed artificial disc imitates the natural mutual motion of the vertebral pair associated with the disc.

It is to be understood that further input as to the desired characteristics of the corrected motion of the patient's spine can be provided by the medical team treating the patient, such as surgeons, neurologists, and physiotherapists. This input may be taken into consideration when determining the stiffnesses of each link, resulting in modification of the stiffnesses to allow more or less motion in a given direction based on the clinical history and projected needs of the patient.

Finally, the procedure followed in steps 60 to 65 results in step 66 in an artificial disc selected having the stiffness values resulting from the preceding steps, the stiffness for each leg being optimized to reflect as closely as possible the relative motion of the natural disc in each direction of motion.

Reference is now made to FIG. 6B, which is a block-diagram flowchart showing the use of inverse and forward kinematics to determine the parameters of an artificial disc for a given patient, such that the replacement disc will imitate to the greatest possible extent, the motions allowed by the natural disc. These steps follow those in the flowchart of FIG. 6A. The input to the system is as follows: Ai and Bi comprise the ends of a line representing the length of each of six individual links reflecting the intervertebral distance at various points on the preoperative medical images with the patient in a resting pose. F is an external load vector from the force values shown in in FIG. 2, q represents the disc dimensions shown in medical images of the patient in various poses of flexion/extension, lateral bending and axial rotation, and n is a force vector where each component accounts for the compressive load in each link.

In step 601, the displacement (d) of each link from its resting length (A,B) is measured from the medical images of the patient taken in each pose (q) of F/E, LB, and AR. In step 606, performed in parallel to step 601, the force (F) on the disc in each position of motion is determined using the Jacobian matrix (J) and the values in FIG. 2. The results from steps 601 and 606, i.e., the displacement (d) of each link in each position of motion and the relevant forces (n) on each link in each position, are then input to step 602, which uses inverse kinematics to derive the stiffness value (linear spring constant, k) for each link in each position of motion. The equivalent (k) of each link for each motion is then given by k=n/d. In step 603, for simplicity, a single, weighted stiffness value for each link is selected as described in FIG. 6A above, taking into account the relative importance of each of the three characteristic spine motions. These weighted stiffness values are then used in the forward kinematics routine, conveniently performed using a forward kinematics solver, in step 604 to determine the displacements of each link that are then used to build the links of the artificial disc. These calculations yield a new pose of the artificial disc for each motion. The integration of this process, i.e., the final stiffnesses and allowable displacements for each link of the passive parallel mechanism that define the replacement disc, are used in step 605 to define the finite screw axis (FSA) for the disc. The result of these calculations and kinematics process is to generate an artificial disc having FSA values as close as possible to the natural FSA values.

It is to be understood that, while forward and inverse kinematics are used in this implementation, alternatively, other methods of force and stiffness calculations could also be used. Further, the stiffness selection method may use an iterative, global optimization method, such that the stiffnesses are chosen to take into account other factors as determined by the patient's medical images and history.

Reference is now made to FIGS. 7 to 10, which illustrate various aspects and methods by which the artificial discs of the resent disclosure can be constructed such that their insertion and implementation can be performed by minimally invasive surgery within a cavity formed by retraction of the tissues in the patient's back.

Reference is first made to FIG. 7, which shows an isometric representation of a completely assembled artificial disc, showing how the links 72 can be connected to the platforms 71, 73 of the disc by means of ball joints 74. Most conveniently, the balls 75 are located at the ends of the links, with the hollows 76 formed in the platforms. The surfaces of the disc 77 contacting the adjacent vertebrae may have ridges 78 or other elements that enable the disc to be firmly positioned between the vertebral body end faces. The engineering problem solved in the exemplary construction shown in FIGS. 7 to 10, shows how the construction of each of the components of these artificial discs enables them to be inserted into a cavity retracted within the tissues of the patient's back, through a minimally invasive incision. Furthermore, the construction of the entire artificial disc itself must be such that it can be assembled within the patient's back from parts inserted minimally invasively.

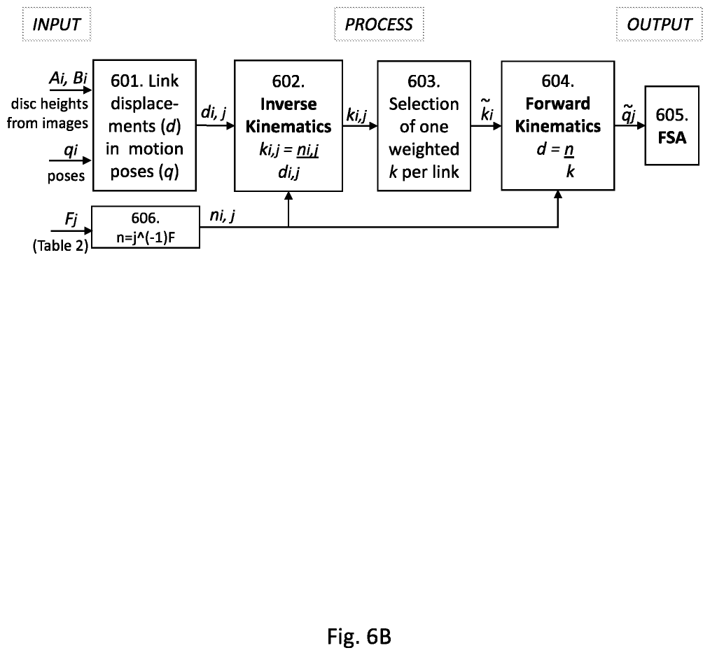

Reference is now made to FIG. 8A, which illustrates a blown up section 79 of the ball joints shown in FIG. 7, to illustrate a construction by which links 82, inserted separately from the platforms 81, can be attached to the platforms in such a way that the ball joints 84 are held firmly within their sockets, while allowing free angular motion. The suggested construction in FIG. 8A shows a wire 85 passing through a link 82, its ball joint 84 and the platform 81. Once the ball of the ball joint 84 of the link 82 has been located relative to the socket 86 of the ball joint in the platform, tightening of the wire will cause the entire ball joint to come together. FIG. 8B-8D show several representative ways in which the wire 85 could be inserted. In FIG. 8B, a single wire 85 is used to pass between all six of the links, which on tightening, assembles all of the links relative to the platform. In FIG. 8C, each link may have a single wire running through it and connecting it to both platforms, one end of the wire having a fixed end 87 within one of the platforms. In FIG. 8D, a third option is that each link may have the fixed ends 87 of two wires within each link 82, and each wire is threaded through a passageway in a separate platform. Other methods of inserting and configuring the wires are also possible.

Although the constructions described above enable assembly of the entire artificial disc within the patient's back, there is still a need to provide a constructional method whereby the platforms can be inserted minimally invasively. The largest dimension of the platforms may be as big as 40 mm. Since such a size is regarded as being unreasonably large for a minimally invasive procedure, there is a need for constructing the platforms in a manner that will enable them to be inserted in smaller parts.

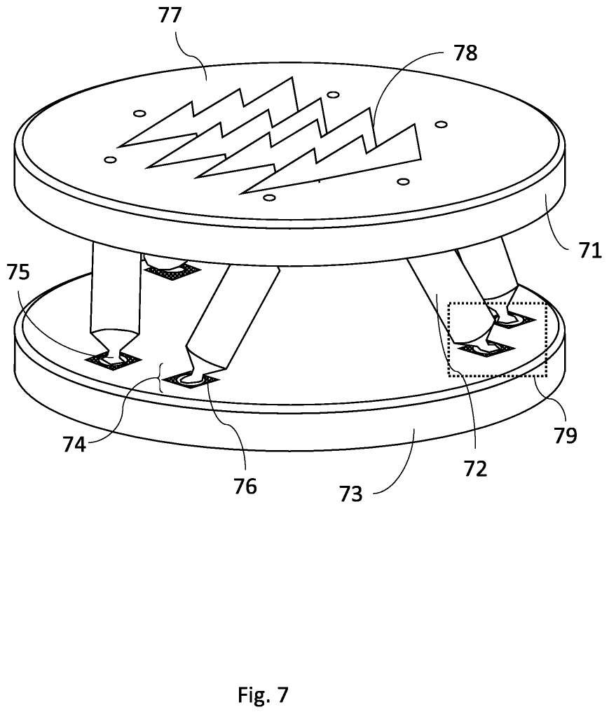

Reference is now made to FIGS. 9A and 9B and FIGS. 10A and 10B, which illustrate two alternative methods by which the platforms can be constructed in smaller parts which can be assembled in situ to generate a rigid plate.

FIGS. 9A and 9B illustrate schematically a first implementation which shows a plate 91 formed of strips 94 each of which is significantly smaller than the overall diameter of the plate. FIG. 9A shows the completely assembled plate 91, with the separate strips 94 firmly attached to each other by means of e.g. dovetail joints 95 along the length of the edges of each strip configured to be joined to a neighboring strip, to form a complete unitary plate 91. FIG. 9B show schematically how the separate strips are dovetailed together. Although a dovetail joint provides good resistance to bending in the direction perpendicular to the length of the joint, FIG. 9B shows an additional feature in the form of a pin 97 or a screw 96 which is inserted through aligned holes in each of the components strips, to ensure even better robustness of the plate construction in the direction perpendicular to the joint length, after the pin 97 or screw 96 has been inserted. A screw 96 has the advantage of providing more positive locking than a pin. However, beyond simple mechanical robustness, the pin 97 or screw 96 also has the function of ensuring that the separate strips are correctly aligned lengthwise relative to each other, thereby ensuring the correct and exact relative locations of the link ball joints, since the accuracy of the planned vertebral motion provided by the disc is only valid if the link joints are in a known predetermined relative position.

FIGS. 10A, 10B, and 10C illustrate schematically a second implementation which shows a platform 10 formed of pie-shaped or pizza-shaped segments or “slices” 14, each of which has a dimension in one direction significantly smaller than the overall diameter of the plate. FIG. 10A shows the completely assembled plate 10 with the separate segments 14 firmly attached to each other by means of dovetail joints along the common interface surfaces between adjacent “slices” of the platform. FIG. 10B shows schematically how the separate segments 14 are dovetailed 15 together to form a unitary plate structure 10. The integrity of the plate has to be ensured by any suitable connection method. One such method shown in FIG. 10C may be by means of two, and preferably more, screws 16 inserted into two or three of the segments 14 at a time, so that all segments are held together by at least one screw 16. Another possible configuration (not shown in FIGS. 10A-10C) is by means of a wire attached around the outer circumference of the plate to ensure that none of the segments can move relative to its neighbors. Such a wire would need to have a high tensile strength, in order to exert the compressive forces onto the segments to prevent them from separating.

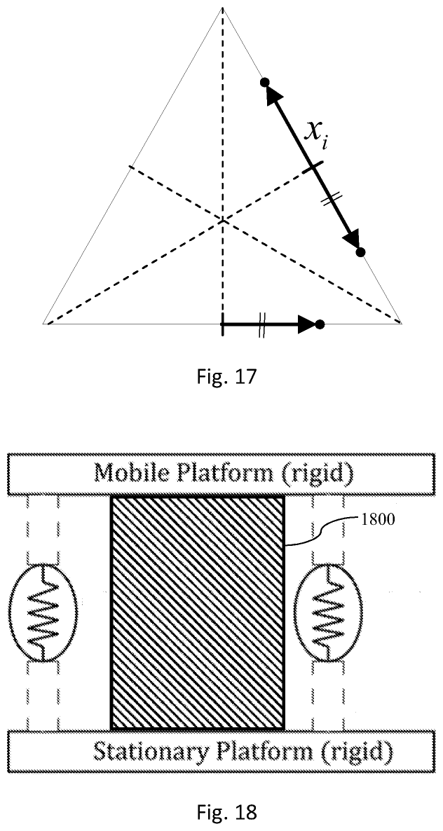

The interior of the personalized intervertebral disc replacement may include a semi-rigid nuclear body comprising inert biocompatible material having a specific pre-calculated resistance for the subject. FIG. 18 illustrates schematically an intervertebral disc replacement that includes a semi-rigid nuclear body 1800. The semi-rigid nuclear body 1800 may be a flexible body that mimics the function of the nucleus pulposus. The semi-rigid nuclear body 1800 may be positioned between the two platforms, and may include, for example, polymers or hydrogels.

The following examples describe some further concepts of the invention with reference to particular examples. The general concepts of the current invention are not limited to the particular examples.

Over the past three decades, numerous new parallel robot configurations have been introduced; see e.g. [1], While the most notable one is the original configuration of the Stewart-Gough platform [2], each new configuration features a unique combination of number of degrees of freedom (DOF), dimensions and actuation that better fit certain applications. For example, the use of a parallel robot as a load sensor requires high measurement sensitivity, and therefore, proximity to singular configurations is encouraged, while use of a manipulator as a machining tool requires high rigidity and avoidance of proximity to singularity as much as possible. Thus far, several task-based designs of parallel robots, such as, task-oriented optimization [3], vibration isolation and dynamic modulus-based design [4], workspace-based design [5], and others, have been published.

The parallel robot/mechanism is also commonly used as an active/passive compliant device. It has been proposed for applications such as an amyotrophic lateral sclerosis (ALS) patient aid [6], remote-center compliance device [7] and a 3 and 6 DOF force sensor [8]. The typical applications of these passive mechanisms depends on their stiffness and therefore, stiffness analysis of parallel mechanisms has been extensively discussed in the literature. Precise modeling, based on CAD-FEA methods, of parallel manipulators stiffness, while considering a broad variety of loads, was discussed in [9-10]. Other investigations have focused on different metrics and visualization tools, such as stiffness mapping [11] and stiffness indices [12].

On the other hand, stiffness synthesis of parallel mechanisms, have been discussed much less. Joint stiffness synthesis for passive parallel mechanisms using screw theory, was discussed and solved in [13-14]. These methods offer an algorithm that determines stiffnesses of springs connected in parallel, in order to realize an arbitrary symmetric positive semi-definite (PSD) stiffness matrix. Simaan and Shoham proposed a method that synthesizes the geometry of a 6 DOF variable geometry parallel robot in order to achieve a certain stiffness matrix [15].

Most of the publications on parallel mechanism stiffness synthesis have focused on general stiffness requirements, such as avoiding singularities, high stiffness condition number and enhancing the overall mechanism stiffness [16-21]. However, to the best of the authors' knowledge, stiffness synthesis of parallel mechanism to achieve a given set of twists for a given set of wrenches, has not been addressed. This task cannot be addressed with the common stiffness synthesis goals, such as stiffness indices and condition numbers, since, in this case, the resulting stiffness matrix is generally not feasible (non PSD). Hence, the present paper suggests an optimization scheme to obtain the set of twists closest to the required ones under a given set of wrenches. As an example, this scheme is demonstrated in the synthesis of an artificial intervertebral spinal disc, where the loads on the spine are given and at the same time, the artificial disc is expected to provide, as closely as possible, the natural motion between two adjacent vertebrae.

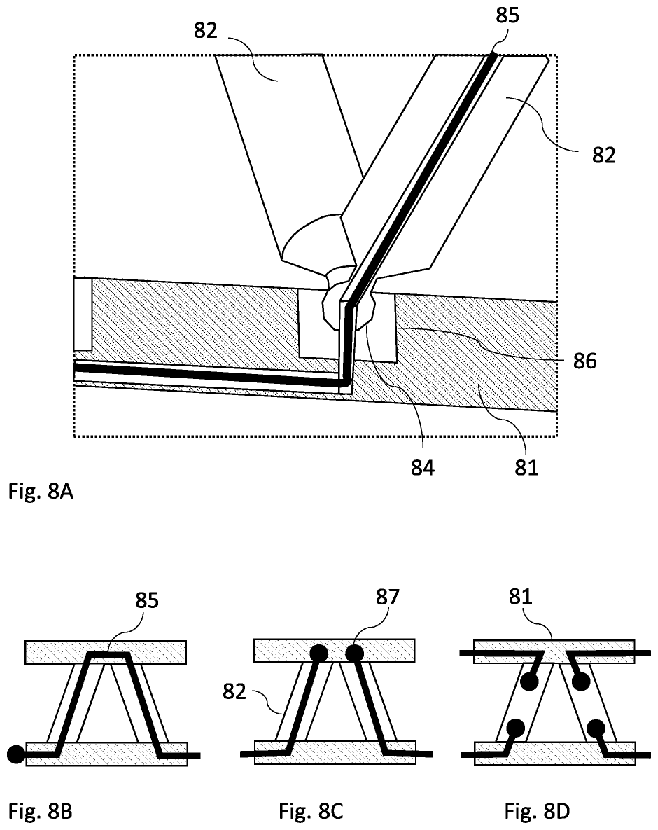

Presented herein is a scheme for construction of the stiffness matrix of a parallel robot for given sets of loads and motion screws. For the synthesis problem, the non-actuated links and platforms are assumed to be rigid, massless and ideal in terms of backlash. The problem is then reduced to the design of actuator stiffnesses, as illustrated in FIG. 11.

A common way to mathematically describe lines is by way of Plücker coordinates:

l=(\hat{n},\vec{m}), (1)

where \hat{n} is a unit vector representing the axis of rotation, and \vec{m} is the moment of vector \hat{n} around the origin of coordinates, which leads to:

|\hat{n}|=1;\hat{n}·\vec{m}=0. (2)

Using line coordinates, a screw can be written as [15]:

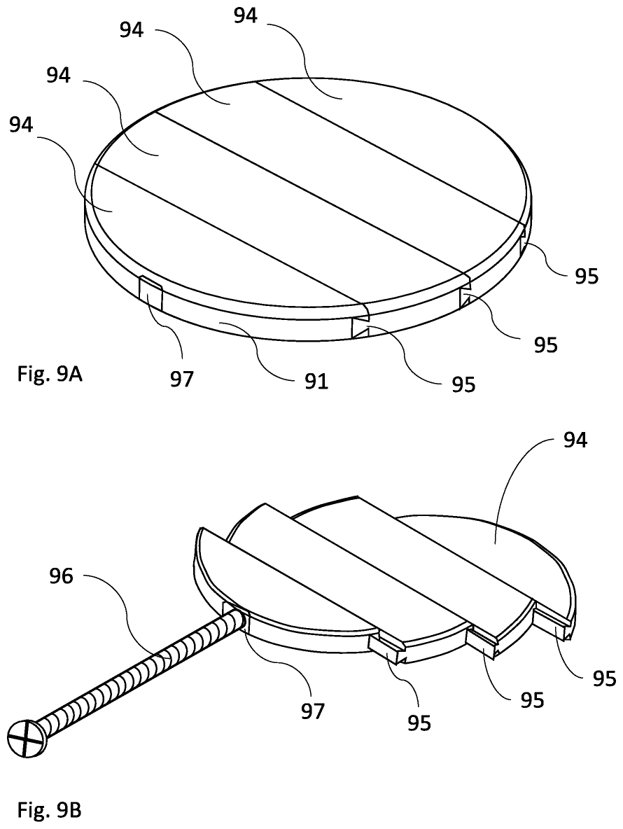

S/≙(\vec{s},\vec{s}₀×\vec{s}+p\vec{s}) (3)

where \vec{s} is a vector along the screw, \vec{s}₀ is a position vector of any point located on the screw, and p is the pitch. FIG. 12 illustrates notations for a rigid body screw motion. A general rigid body motion is described by 6 coordinates:

$$Δ x = [ \begin{matrix}Δ {r}_{x} & Δ {r}_{y} & Δ {r}_{z} & Δ {θ}_{x} & Δ {θ}_{y} & {Δ {θ}_{z} ]}^{T} = [ \begin{matrix}Δ \vec{r} \\ Δ \vec{θ}\end{matrix} ] ,\end{matrix} \qquad ( 4 )$$

or as a screw:

S/=ϕ(\vec{s},\vec{s}₀×\vec{s}+p\vec{s}), (5)

where ϕ is the rotation angle around the body's axis of rotation \vec{s}. Recall that p=t/ϕ, where t is the translation along \vec{s}; the rigid body motion represented by a screw is:

S/≙(ϕ\vec{s},ϕ\vec{s}₀×\vec{s}+t\vec{s}). (5)

When forcing a certain screw motion upon a rigid body, then from (4) and (6), the displacement is:

$$\{ \begin{matrix}Δ \vec{r} = ϕ {\vec{s}}_{0} \times \vec{s} + t \vec{s} \\ Δ \vec{θ} = Rot ( ϕ , \vec{s} )\end{matrix} . \qquad ( 7 )$$

The stiffness matrix is defined as the ratio of a load to the resulting set of displacement:

F=KΔx (8)

where F is an external load vector, K is the stiffness matrix of the robot, and Δx is the displacement vector of the moving platform.

The stiffness matrix is a local entity and hence, relates the applied loads to an infinitesimal (not finite) screw. In the sequel, Δx is considered to be infinitely small and therefore, K becomes the ratio of the applied wrench to the resulting twist.

In our case, one looks to synthesize K where both the applied wrench and the resulting twist are given. In a set of twists ΔX [symbol] [Δx₁ . . . Δxₙ], where column i is twist i and a corresponding set of wrenches F [symbol] [F₁ . . . Fₙ], the desired stiffness matrix becomes:

F=K_{req}ΔX, (9)

where K_{req} is the required stiffness matrix given by:

K_{req}=F(ΔX)^{T}(ΔX(ΔX)^{T})⁻¹. (10)

However, the required stiffness matrix K_{req} obtained from (10) is a 6×6 matrix and, in general, does not exhibit any special characteristics such as non-singularity, symmetry and positive definitiveness—necessary characteristics of a static stiffness matrix when external loads are absent [22].

Let us assume that the parallel robot shown schematically in FIG. 11, includes rigid links, whereas the actuators between the two plates are the only non-rigid elements. When assuming the same stiffness for each actuator, the stiffness matrix is [11]:

K=kJ^{T}J, (11)

where k>0 is a scalar representing the stiffness of each actuator, and J is the Jacobian matrix of the parallel robot, which satisfies:

J\dot{x}=\dot{q}. (12)

Equation (9) can be extended to represent the case of different actuator stiffnesses. Let us assume that actuators 1, . . . , m have a stiffness of k₁, . . . , kₘ, respectively. The ratio between the actuator's infinitesimal load and displacements is:

δn=K̅δq (13)

where K̅=diag{k₁, . . . ,kₘ} is a diagonal matrix of the actuator stiffnesses and n is the actuator forces vector. For the identical stiffness case, this matrix is reduced to K̅=kI.

Therefore, eq. (11) can be extended to:

K=J^{T}K̅J (14)

Due to the fact that actuator stiffness is always positive, it is obvious that although each actuator has a different stiffness, the overall stiffness matrix is symmetric and PSD.

Obtaining a non-symmetric stiffness matrix of a parallel robot has barely been discussed in the literature, as it was presented only under additional external load [22]. The present investigation aims to select the actuator stiffnesses of a parallel robot in order to synthesize a feasible stiffness matrix for a given sets of load wrenches and corresponding motion twists. In particular, this section presents the selection process of a set of actuator stiffnesses k₁, . . . , kₘ, to obtain a feasible, symmetric, PSD stiffness matrix that optimally resembles the given motion under the given load.

A screw can be represented by a line in a 3D space, with a certain pitch and rotation amplitude. Therefore, the variance between two screws can be quantified by four scalar parameters: Δd—the distance between the screw lines, Δα—the spatial angle between the screw lines, Δϕ—the rotation angle difference along the line and Δt—the translation difference along the screws. In this paper, the variance between two screws, i.e., the given required twist, versus the twist obtained from the feasible stiffness matrix, is minimized.

In order to avoid multi-objective optimization routines, there is a need to formulate a single cost function that includes all of these difference parameters. However, Δd and Δt quantify lengths, while Δα and Δϕ are angles and are therefore dimensionless. In order to make the quantities dimensionally homogenous, Δd and Δt are normalized by a characteristic length L:

Δd*=Δd/L;Δt*=Δt*/L (15)

In order to allow the cost function to compare several pairs of screws, the difference parameters are augmented into n-space vectors: a distance vector Δ\vec{d}*=[Δd₁* . . . Δdₙ*]^{T}, a translation difference vector Δ\vec{t}*=[Δt₁* . . . Δtₙ*]^{T}, an angle difference vector Δ\vec{α}=[Δα₁ . . . Δαₙ]^{T}, and a rotation difference vector Δ\vec{ϕ}=[Δϕ₁ . . . Δϕₙ]^{T}.

The cost function can then be formulated as a sum of four quadratic terms:

[symbol]

[symbol]

\vec{d}*^{T}W_{d}Δ\vec{d}*+Δ\vec{α}^{T}W_{α}Δ\vec{α},+Δ\vec{ϕ}^{T}W_{ϕ}Δ\vec{ϕ}+Δt*^{T}WₜΔt* (16)

where W_{d}, W_{α}, W_{ϕ}, Wₜ∈ [symbol] ^{n×n} are positive semi-definite weights. As the importance of each of the four weights may vary between application, they are user-tuned to match their importance. For example, selection of W_{d}, W_{α}»W_{ϕ}, Wₜ emphasizes a greater significance of preserving the position and orientation of the screw lines, while selection of W_{ϕ}»W_{d}, W_{α}, Wₜ emphasizes a rotational range of motion preservation with little to no consideration of the axis of rotation.

Let us assume a robot with a fixed, known geometry. The design goal is to minimize the difference between the required and the actual screws, represented by the cost function mentioned above. In this paper, the design parameters are the actuator stiffnesses, which are always positive. Therefore, the problem can be formulated as a nonlinear minimization problem with m linear constraints:

$$\{ \begin{matrix}{min}_{{k}_{i}} 𝒥 = 𝒥 ( {k}_{1} , … , {k}_{m} ) \\ s . t . {k}_{i} > 0\end{matrix} \qquad ( 17 )$$

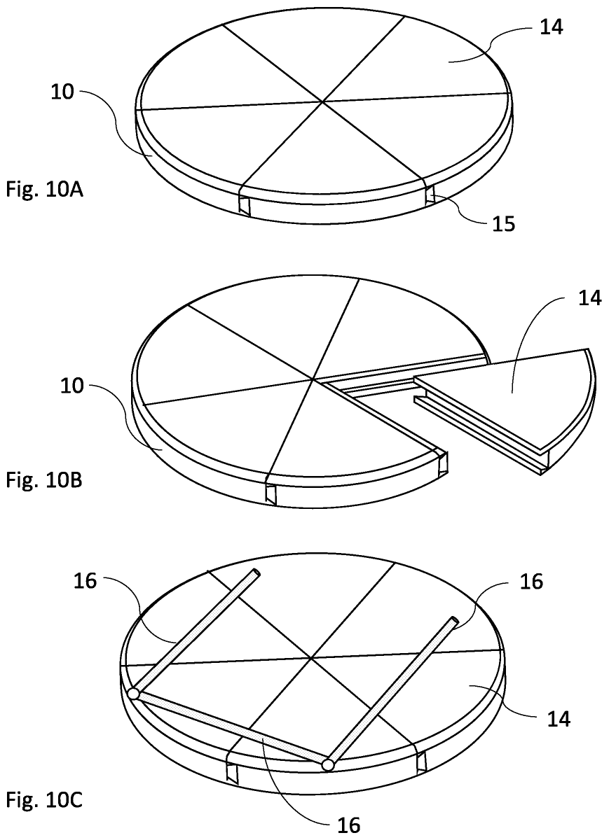

Even though [symbol] is quadratic, obtaining and analyzing the closed form expression for [symbol] (k₁, . . . , kₘ) is complicated, and there is no guarantee that the problem is convex, and in fact, simulation has yielded a large amount of local minimum points. Therefore, a simple local optimization algorithm is insufficient, as there is a need for a global optimization method. The calculation process of [symbol] for each iteration (a set of stiffnesses) is depicted in FIG. 13. Iteration j begins with a set of stiffnesses kᵢ^{(j)}, and the stiffness matrix K^{(j)} of the robot is calculated using (14). The resulting displacements of the platform are then calculated:

ΔXᵣₑₛ=(K^{(j)})⁻¹F (18)

where F is the load matrix. The resulting screw parameters are then extracted from ΔXᵣₑₛ, and cost function [symbol] is then re-calculated.

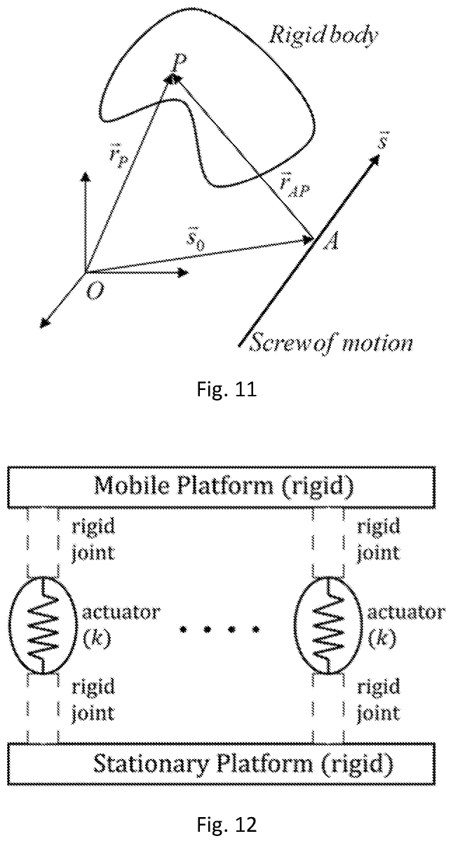

Multi start scatter search is a nonlinear programming algorithm used for global optimization problems[23]. It runs several local optimization routines from different starting points, and compares the resulting minimal cost in order to obtain the global minimum from the local ones [24] [25]. The overall scheme is drawn in FIG. 14.

The process starts by running a local optimization routine from an initial guess kᵢ⁽⁰⁾. Once convergence is achieved, a set of random trial points is generated, and each point is given a score, which consists of the cost function value at the point and a multiple of the sum of constraints violations. Another local optimization routine is then initialized from the point with the best score. After two local optimization solutions are obtained, a basin of attraction is defined for each local minimum. The basin of attraction is assumed to be a sphere centered in the local solution, with the distance to the initial condition as a radius.

The next steps are iterative—each of the remaining trial points is re-evaluated for its score and proximity to previously found basins of attraction, and a local optimization is initiated from the best point. After each iteration, the basins of attraction are re-evaluated and the process is repeated. Once all of the trial points have been tested, the best local minimum found is selected as the global minimum of the cost function.

The routine is implemented using MATLAB optimization toolbox command ‘GlobalSearch’ with 5000 trial points.

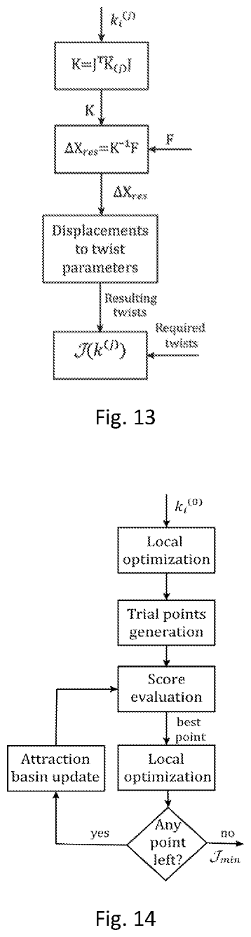

The stiffness synthesis algorithm is validated by a design problem in which the load and the motion are simultaneously provided. This is the case in designing an artificial intervertebral spinal disc, since both the kinematic behavior of adjacent vertebrae, as well as the loads applied, are predefined. An artificial intervertebral disc which consists of a passive parallel robot of the Stewart-Gough type (FIGS. 5A, 15), is used here as an illustrative example. The design of an artificial disc often includes an attempt to preserve the natural motion. In the case of the disc joints, the natural motion is most accurately characterized by instantaneous twist or finite screw [26] [27]. For the numerical simulation, the dimension of size, displacements and loads are taken from [28] [29]. The base and mobile platform actuator coordinates are specified in Table V, where A represents the base coordinates and B represents the platform coordinates.

Table VI and Table VII list the loads and matching displacements of a natural disc joint that were used to construct the displacement screws, respectively. In all cases, the initial pose of the moving platform (with reference to the world coordinate system) was x=[0 0 12 0 0 0]^{T} (all lengths are in millimeters), while the stationary platform is centered in the origin. As mentioned in section III, there is a need to normalize the lengths in order to achieve a dimensionally-homogenous cost function. All lengths are normalized with the characteristic length, which is the L4/L5 level disc height of L=12 (mm)[28].

Let us first discuss the naïve design approach. The spinal motion consists of six primary motions: flexion/extension, lateral bending (left and right) and axial rotation (left and right). Since the wrenches applied at the adjacent vertebra for each one of these motions are known, the same wrenches can be applied on the moving platform of the artificial disc, enabling calculation of the resulting force at each leg of the parallel robot. Given the initial and final pose of adjacent vertebrae, one also obtains the elongation of each leg of the parallel robot, and hence, the stiffness of each leg for each motion. In order to obtain a single stiffness value for each leg, the stiffnesses obtained from all motions for the same leg are then averaged, and used to calculate the parallel robot stiffness matrix using Eq. (9). Table I provides the variance between the required screw and the one provided by the artificial disc joint, with average stiffness for each leg, as calculated above.

TABLE 1
SCREW DIFFERENCES FOR THE NON-OPTIMAL
(AVERAGE STIFFNESS) CONFIGURATION

| | | | Δt | |
|---|---|---|---|---|
| | Δd (mm) | Δα(°) | (mm) | Aϕ (°) |
| Flexion | 7.56 | 4.71 | 0.01 | 4.78 |
| Extension | 6.96 | 5.65 | 0.01 | 3.10 |
| Right | 8.91 | 7.04 | 0.01 | 3.95 |
| Bending | | | | |
| Left | 9.26 | 8.24 | 0.04 | 3.95 |
| Bending | | | | |
| Right | 0.06 | 14.31 | 0.02 | 0.52 |
| Torsion | | | | |
| Left | 0.10 | 15.20 | 0.01 | 0.52 |
| Torsion | | | | |

Table II lists the screw parameter differences for each spinal motion (6 in total) obtained as a result of the optimization process that minimizes the variance between the required and the actual motion, defined as screws. For a preliminary optimal solution, all weights are selected to be unit matrices: W_{d}, W_{α}, W_{ϕ}, Wₜ=I₆. It can be observed that the screw differences vary between motions: for axial torsion, screws are rotated by twice the amount than for bending, and by an order of magnitude more than flexion and extension, while the distance between the screws is smaller by an order of magnitude for flexion and extension than for the rest of the motions.

TABLE II
SCREW DIFFERENCES FOR THE OPTIMAL CONFIGURATION
WITH UNIT WEIGHTS FOR EACH DIRECTION OF MOTION

| | | | Δt | |
|---|---|---|---|---|
| | Δd (mm) | Δα(°) | (mm) | Aϕ (°) |
| Flexion | 0.32 | 0.05 | <0.01 | 4.96 |
| Extension | 2.63 | 1.00 | <0.01 | 3.18 |
| Right | 12.00 | 7.99 | 0.03 | 2.48 |
| Bending | 12.03 | 5.24 | 0.01 | 2.48 |
| Left | | | | |
| Bending | 9.33 | 14.80 | 0.01 | 2.07 |
| Right | | | | |
| Torsion | 9.14 | 14.88 | 0.02 | 2.08 |
| Left | | | | |
| Torsion | | | | |

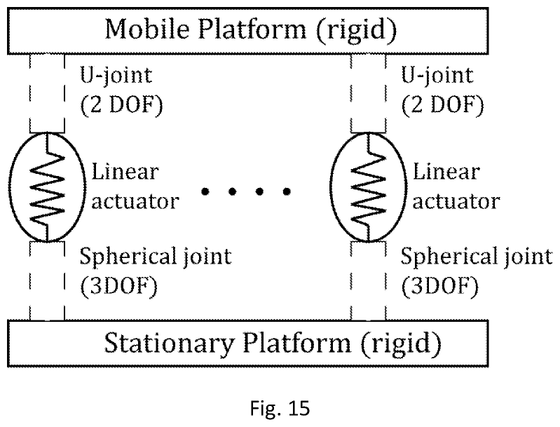

Let us now assume that there is a need to minimize the line distances Δd and Δϕ (for range of motion preservation) while focusing more on preserving the lateral bending. Therefore, the relative weights of Δd and Δϕ, as well as the relative weights of lateral bends, are increased. The weights are now: (lateral bends are motions no. 3 and 4) W_{d}=144W, W_{α}=W, W_{ϕ}=20W, Wₜ=W, where W=diag{1,1,5,5,1,1}. Numerical results are listed in Table III. It can be clearly observed that typical Δd and Δϕ decreased on the expense of an increase in Δt and Δα, caused by the change in relative magnitudes of the weights. It can also be observed that due to the greater significance given to lateral bendings, the distance and rotation magnitude for these motions decreased the most, while line angles increased by only 2°. As Δt had the smallest weights and typical magnitudes, they displayed the largest increase, of up to 400% of magnitudes from case II. The resulting twist lines are plotted in FIG. 16. Although the optimized solution yielded slightly worse results for axial torsion, it had significantly improved results for flexion/extension, and slightly improved results for lateral bending, which is the design goal of the process. As seen in Table IV and Table. V, flexion and extension motions tend to have a larger range of motion, which translates into smaller stiffnesses, and therefore, the relative effect of these motions on the overall stiffness can be accidentally eliminated in case of averaging and failure to optimize the leg stiffnesses, a phenomenon that is prevented with the proposed method. While the desired screw parameters differences have decreased other parameters have increased—this phenomenon occurs due to the inherit differences between the motions. As axial torsion requires the stiffness of every link to be relatively the same due to same distances of each link from the screw axis, lateral bending and flexion/extension requires great variance in link stiffnesses due to greater variance in distances from the screw.

TABLE III
SCREW DIFFERENCES FOR THE OPTIMAL CONFIGURATION
WITH EXTRA WEIGHTS ON LATERAL BENDING

| | | | Δt | |
|---|---|---|---|---|
| | Δd (mm) | Δα(°) | (mm) | Aϕ (°) |
| Flexion | 0.97 | 1.05 | 0.39 | 3.99 |
| Extension | 1.54 | 1.84 | 0.17 | 2.75 |
| Right | 8.11 | 9.74 | 0.36 | 1.35 |
| Bending | | | | |
| Left | 6.40 | 7.28 | 0.41 | 1.35 |
| Bending | | | | |
| Right | 4.93 | 15.20 | 0.21 | 4.56 |
| Torsion | | | | |
| Left | 4.46 | 15.77 | 0.18 | 4.56 |
| Torsion | | | | |

TABLE VI
NATURAL DISPLACEMENTS
FOR VERTEBRAL MOTION

| | rₓ | r_{y} | r_{z} | θₓ | θ_{y} | θ_{z} |
|---|---|---|---|---|---|---|
| | (mm) | (mm) | (mm) | (°) | (°) | (°) |
| Flexion | 0.03 | 0.2 | 0.03 | −5.9 | 0.2 | 0.4 |
| Extension | −0.02 | −0.1 | −0.07 | 3.6 | −0.4 | 0 |
| Left | −0.17 | 0.04 | −0.05 | 0.8 | −4.7 | −0.3 |
| Bending | | | | | | |
| Right | 0.16 | −0.03 | −0.00 | −0.2 | 5 | −0.1 |
| Bending | | | | | | |
| Left | 0.06 | −0.03 | −0.00 | 0.6 | 0.1 | 2.3 |
| Twisting | | | | | | |
| Right | −0.06 | −0.03 | −0.00 | 0.6 | −0.1 | −2.3 |
| twisting | | | | | | |

TABLE V
SELECTED LOADS FOR VERTEBRAL MOTION

| | Fₓ | F_{y} | F_{z} | Mₓ | M_{y} | M_{z} |
|---|---|---|---|---|---|---|
| | (N) | (N) | (N) | (N · m) | (N · m) | (N · m) |
| Flexion | 0 | 0 | −400 | −10.6 | 0 | 0 |
| Extension | 0 | 0 | −400 | 4.7 | 0 | 0 |
| Left | 0 | 0 | −400 | 0 | −10.6 | 0 |
| Bending | | | | | | |
| Right | 0 | 0 | −400 | 0 | 10.6 | 0 |
| Bending | | | | | | |
| Left | 0 | 0 | −400 | 0 | 0 | 10.6 |
| Twisting | | | | | | |
| Right | 0 | 0 | −400 | 0 | 0 | −10.6 |
| twisting | | | | | | |

However, even with those limiting factors that are specific for this problem and this specific robot geometry, overall performance was satisfying: it was shown that all of the relative distances of the screw axes were smaller than the characteristic length of the problem. Relaxing the fixed-geometry assumption may yield improved results, as discussed below.

Presented herein is a novel method for parallel robot stiffness synthesis in the case of given both loads as well as required motion. The resulting stiffness matrix is generally not feasible since the ratio of the loads to motions might result in an asymmetrical and non-PSD matrix. To obtain a valid stiffness matrix, the variance between the screw parameters of the required versus the actual ones is minimized. The synthesis problem is formulated as a nonlinear optimization problem, with actuator stiffnesses as optimization variables, and a cost function that quantifies the screw variance.

Due to the non-convexity of the problem, a nonlinear programming solver was needed, and the multi start algorithm was used, and implemented in MATLAB.

The synthesis of an optimized valid stiffness matrix was demonstrated by design of an artificial intervertebral spinal disc joint, since in this case, the natural motion should be preserved under a given set of loads. A parallel robot consisting of a passive Stewart-Gough platform, replaced the natural disc, and its legs stiffnesses were optimized to resemble, as close as possible, the natural motion of the spine. The optimization cost function was comprised of the variance between the required screw motions and the actual ones obtained from a valid stiffness matrix.

For applications other than the artificial spinal disc brought here, it is possible to weigh the screw parameters differently, to emphasize the significance of specific motions needed to be maintained.

In this investigation, the parallel robot kinematic structure and dimensions were assumed to be fixed. One possible approach to obtain even better resemblance of the required motion is by relaxing the assumptions of a given kinematic structure as well as fixed dimensions of the parallel robot. However, one must consider passage through singular configurations while using iterative architecture-generating algorithms.

### REFERENCES

[1] J. Merlet, Parallel robots. Kluwer Academic Publishers, Durdrecht, 2000.
[2] D. Stewart, “A Platform with Six Degrees of Freedom,” Proc. Inst. Mech. Eng., vol. 180, no. 1, pp. 371-386, June 1965.
[3] A. Wolf and M. Shoham, “Screw theory tools for the synthesis of the geometry of a parallel robot for a given instantaneous task,” Mech. Mach. Theory, vol. 41, no. 6, pp. 656-670, 2006.
[4] J. T. Xing, Y. P. Xiong, and W. G. Price, “Passive-active vibration isolation systems to produce zero or infinite dynamic modulus: Theoretical and conceptual design strategies,” J. Sound Vib., vol. 286, no. 3, pp. 615-636, 2005.
[5] C. M. Gosselin and M. Guillot, “The Synthesis of Manipulators with Prescribed Workspace,” J. Mech. Des., vol. 113, no. 4, p. 451, December 1991.
[6] H. Zhang, K. Albee, and S. K. Agrawal, “A spring-loaded compliant neck brace with adjustable supports,”Mech. Mach. Theory, vol. 125, pp. 34-44, July 2018.
[7] H. McCallion, G. R. Johnson, and D. T. Pham, “COMPLIANT DEVICE FOR INSERTING A PEG IN A HOLE.,” Ind. Rob., vol. 6, no. 2, pp. 81-87, February 1979.
[8] T. A. Dwarakanath, B. Dasgupta, and T. S. Mruthyunjaya, “Design and development of a Stewart platform based force-torque sensor,” Mechatronics, vol. 11, no. 7, pp. 793-809, 2001.
[9] A. Klimchik, “Enhanced stiffness modeling of serial and parallel manipulators for robotic based processing of high performance materials,” Ecole Centrale de Nantes (ECN); Ecole des Mines de Nantes, 2011.
[10] A. Pashkevich, D. Chablat, and P. Wenger, “Stiffness analysis of overconstrained parallel manipulators,” Mech. Mach. Theory, vol. 44, no. 5, pp. 966-982, 2009.
[11] C. Gosselin, “Stifness Mapping for Parallel Manipulators,” IEEE Trans. Robot., vol. 6, no. 3, pp. 377-382, 1990.
[12] G. Carbone and M. Ceccarelli, “Comparison of indices for stiffness performance evaluation,” Front. Mech. Eng. China, vol. 5, no. 3, pp. 270-278, 2010.
[13] R. G. Roberts, “Minimal realization of a spatial stiffness matrix with simple springs connected in parallel,” IEEE Trans. Robot. Autom., vol. 15, no. 5, pp. 953-958, 1999.
[14] S. Huang and J. M. Schimmels, “Achieving an Arbitrary Spatial Stiffness with Springs Connected in Parallel,” J. Mech. Des., vol. 120, no. 4, pp. 520-526, December 1998.
[15] M. S. C. Yuan and F. Freudenstein, “Kinematic Analysis of Spatial Mechanisms by Means of Screw Coordinates. Part 1—Screw Coordinates,” J. Eng. Ind, vol. 93, no. 1, pp. 61-66, February 1971.
[16] D. Chablat and P. Wenger, “Architecture optimization of a 3-DOF translational parallel mechanism for machining applications, the orthoglide,” IEEE Trans. Robot. Autom., vol. 19, no. 3, pp. 403-410, 2003.
[17] A. Pashkevich, P. Wenger, and D. Chablat, “Design strategies for the geometric synthesis of Orthoglide-type mechanisms,” Mech. Mach. Theory, vol. 40, no. 8, pp. 907-930, 2005.
[18] C. S. Long, J. A. Snyman, and A. A. Groenwold, “Optimal structural design of a planar parallel platform for machining,” Appl. Math. Model., vol. 27, no. 8, pp. 581-609, August 2003.
[19] E. Courteille, D. Deblaise, and P. Maurine, “Design optimization of a Delta-like parallel robot through global stiffness performance evaluation,” in 2009 IEEE/RSJ International Conference on Intelligent Robots and Systems, 2009, pp. 5159-5166.
[20] X. J. Liu, Z. L. Jin, and F. Gao, “Optimum design of 3-DOF spherical parallel manipulators with respect to the conditioning and stiffness indices,” Mech. Mach. Theory, vol. 35, no. 9, pp. 1257-1267, September 2000.
[21] A. Moosavian and F. Xi, “Design and analysis of reconfigurable parallel robots with enhanced stiffness,” Mech. Mach. Theory, vol. 77, pp. 92-110, 2014.
[22] M. Griffis and J. Duffy, “Global stiffness modeling of a class of simple compliant couplings,” Mech. Mach. Theory, vol. 28, no. 2, pp. 207-224, 1993.
[23] DIXON and L. C. W., “The Global Optimization Problem. An Introduction,” Towar. Glob. Optim., vol. 2, pp. 1-15, 1978.
[24] Z. Ugray, L. Lasdon, J. Plummer, F. Glover, J. Kelly, and R. Marti, “Scatter search and local NLP solvers: A multistart framework for global optimization,” INFORMS J. Comput., vol. 19, no. 3, pp. 328-340, 2007.
[25] F. Glover, “A template for scatter search and path relinking,” in Artificial Evolution, 1998, pp. 1-51.
[26] N. Bogduk, B. Amevo, and M. Pearcy, “A biological basis for instantaneous centres of rotation of the vertebral column,” Proc. Inst. Mech. Eng. Part H J. Eng. Med., vol. 209, no. 3, pp. 177-183, 1995.
[27] M. J. Pearcy and N. Bogduk, “Instantaneous axes of rotation of the lumbar intervertebral joints,” Spine (Phila. Pa. 1976)., vol. 13, no. 9, pp. 1033-1041, 1988.
[28] S. H. Zhou, I. D. McCarthy, A. H. McGregor, R. R. Coombs, and S. P. Hughes, “Geometrical dimensions of the lower lumbar vertebrae—analysis of data from digitised CT images.,” Eur. Spine J., vol. 9, no. 3, pp. 242-8, 2000.
[29] A. B. Schultz, D. N. Warwick, M. H. Berkson, and A. L. Nachemson, “Mechanical Properties of Human Lumbar Spine Motion Segments—Part I: Responses in Flexion, Extension, Lateral Bending, and Torsion,” J. Biomech. Eng., vol. 101, no. 1, pp. 46-52, February 1979.
[30] H. Bamberger, M. Shoham, and A. Wolf, “Kinematics of micro planar parallel robot comprising large joint clearances,” Advances in Robot Kinematics. pp. 75-84, 2006.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

1. A personalized intervertebral disc replacement for a subject, comprising:
a first element adapted to contact a first vertebra in the spine of said subject;
a second element adapted to contact a second vertebra adjacent to said first vertebra in the spine of said subject; and
a set of links coupling said first and second elements, said links arranged as a passive parallel mechanism, each of said links having a predetermined stiffness and length.

2. The personalized intervertebral disc replacement for a subject of claim 1, wherein at least some of said links are oriented obliquely to a direction perpendicular to either of said first and second elements.

3. The personalized intervertebral disc replacement for a subject of claim 1, wherein an interior of the personalized intervertebral disc replacement contains a semi-rigid nuclear body comprising inert biocompatible material having a specific pre-calculated resistance for the subject.

4. The personalized intervertebral disc replacement for a subject of claim 1, wherein said predetermined length and stiffness of each of the links is determined based on preoperative images of said spine of said subject.

5. The personalized intervertebral disc replacement for a subject of claim 1, wherein said predetermined stiffness of each of the links is determined based on spinal load calculations and preoperative medical images of said spine.

6. The personalized intervertebral disc replacement for a subject of claim 5, wherein said preoperative medical images are preoperative medical images of said spine in positions of at least some of erect standing, flexion, extension, lateral bending and axial rotation.

7. The personalized intervertebral disc replacement for a subject of claim 1, wherein said predetermined stiffness of each of the links is determined based on spinal load calculations and preoperative medical images of said spine in positions of erect standing, flexion, extension, lateral bending and axial rotation.

8. The personalized intervertebral disc replacement for a subject of claim 1, having six links.

9. The personalized intervertebral disc replacement for a subject of claim 1, wherein said first and second elements and said links are configured such that they can be inserted individually into the subject and assembled in situ, in a minimally invasive procedure.

10. The personalized intervertebral disc replacement for a subject of claim 1, wherein said stiffnesses are determined using inverse and forward kinematics analysis.

11. The personalized intervertebral disc replacement for a subject of claim 1, wherein said stiffnesses are determined using inverse kinematics and a stiffness matrix.

12. The personalized intervertebral disc replacement for a subject of claim 1, wherein said passive parallel mechanism is in the form of a Stewart-Gough platform.

13. The personalized intervertebral disc replacement for a subject of claim 1, wherein said first and second elements are planar.

14. The personalized intervertebral disc replacement for a subject of claim 1, wherein dynamic motion of said disc is determined according to at least one of a biomechanical atlas, big data, artificial intelligence, and statistical analysis.

15. A method of personalizing an artificial intervertebral disc replacement for a subject, comprising:
acquiring a set of preoperative medical images of said subject's spine;
creating a disc replacement in the form of a passive parallel mechanism having extendable links;
determining a length of each of said links using said medical images of said spine of said subject; and
using inverse and forward kinematics analysis, predetermined spinal load calculations, and said set of preoperative medical images to determine stiffnesses of said links.

16. The method of claim 15, wherein said preoperative medical images include preoperative medical images of said subject's spine in at least some positions of erect standing, flexion, extension, lateral bending and axial rotation.

17. The method of claim 15, wherein said stiffnesses of said links are determined such that dynamic motion of said disc resembles natural motion of the disc of said subject.

18. The method of claim 15, wherein the stiffness of each link is calculated with the assistance of at least one of big data, statistical analysis, and artificial intelligence.

19. The method of claim 15, wherein said passive parallel mechanism is in the form of a Stewart-Gough platform.

20. The method of claim 15, wherein the dynamic motion of said disc is determined according to at least one of a biomechanical atlas, big data, artificial intelligence, and statistical analysis.

21. A modular intervertebral disc replacement for a subject, comprising:
a first plate-shaped element shaped and adapted to contact a first vertebra in the spine of said subject;
a second plate-shaped element shaped and adapted to contact a second vertebra adjacent to said first vertebra in the spine of said subject; and
a set of links adapted to couple said first and second plate-shaped elements as a passive parallel mechanism,
wherein each of said first plate-shaped element and said second plate-shaped element are constructed of a plurality of separate parts adapted to fit together to form their plate-shaped element, each of said separate parts having at least one of its lateral dimensions substantially smaller than lateral dimensions of said plate-shaped elements, said lateral dimensions of said parts, and radial dimensions of said links being sufficiently small that said parts and said links can be inserted separately and minimally invasively into said subject, for assembly in situ in the spine of the subject.

22. The intervertebral disc replacement for a subject of claim 21, wherein said separate parts have joining features which enable them to form a rigid plate-shaped element when joined.

23. The intervertebral disc replacement for a subject of claim 22, wherein said joining features comprise a joint structure along common edges of said parts to be joined, said joint structure providing a robust plate-shaped element when implemented.

24. The intervertebral disc replacement for a subject of claim 23, wherein said joint structure is a dovetail joint.

25. The intervertebral disc replacement for a subject of claim 21, further comprising either a pin or a screw adapted to join said parts to form a rigid plate-shaped element when inserted.

26. The intervertebral disc replacement for a subject of claim 21, wherein said parts are in a form of lateral slices of said plate-shaped element.

27. The intervertebral disc replacement for a subject of claim 21, wherein said parts are in a form of pie-shaped segments of said plate-shaped element.

28. The intervertebral disc replacement for a subject of claim 21, wherein at least one of said links has a longitudinal bore passing through at least part of its length, said bore being adapted to receive a wire, said wire further passing through one plateshaped element, such that tensioning of said wire couples said plate to said link.

29. The intervertebral disc replacement for a subject of claim 28, wherein said wire passes through at least one link and both plate-shaped elements, such that tensioning of said wire couples said plates to said at least one link.

30. The intervertebral disc replacement for a subject of claim 28, wherein more than one wire passes through at least one link and at least one plate-shaped element, such that tensioning of said wires couples said plates to said at least one link.

31. The intervertebral disc replacement for a subject of claim 21, wherein said parts may be inserted into an intervertebral space of said subject through an incision less than half a diameter of the vertebrae.
